(19) United States
(12) Patent Application Publication
(10) Pub. No.: US 2005/0136100 A1
(43) Pub. Date: Jun. 23, 2005

(54) Hollow anti-microbial fibers and fibrous products

(75) Inventor: Stephen W. Foss, Rye Beach, NH (US)

Correspondence Address: PERKINS, SMITH & COHEN LLP, ONE BEACON STREET, 30TH FLOOR, BOSTON, MA, 02108, US

(73) Assignee: Foss Manufacturing Co., Inc., Hampton, NH (US)

(21) Appl. No.: 10/989,961

(22) Filed: Nov. 16, 2004

Related U.S. Application Data
(63) Continuation-in-part of application No. 10/762,920, filed on Jan. 22, 2004.
Application No. 10/762,920 is a division of application No. 09/565,138, filed on May 5, 2000, now Pat. No. 6,723,428.
Application No. 10/762,920 is a continuation-in-part of application No. 09/565,138, filed on May 5, 2000, now Pat. No. 6,723,428.
(60) Provisional application No. 60/136,261, filed on May 27, 1999.
(60) Provisional application No. 60/173,207, filed on Dec. 27, 1999.
(60) Provisional application No. 60/172,285, filed on Dec. 17, 1999.
(60) Provisional application No. 60/172,533, filed on Dec. 17, 1999.
(60) Provisional application No. 60/180,536, filed on Feb. 7, 2000.
(60) Provisional application No. 60/181,251, filed on Feb. 9, 2000.
(60) Provisional application No. 60/180,240, filed on Feb. 4, 2000.

(51) Int. Cl. B32B 5/02; A61K 33/38; A61K 33/42; A61K 9/70

(52) U.S. Cl. 424/443; 424/618; 424/603; 442/123

(57) ABSTRACT

Anti-microbial and/or anti-fungal synthetic hollow fiber (2) and various products made partially or wholly therefrom are formed in pure hollow or mock-hollow shapes and composed of various thermoplastic polymers having dispersed therein organic or inorganic, antimicrobial additives. The thickness of the fiber walls are optimally equal to or slightly less than the average maximum dimensions of the anti-microbial additive particles. Thus, a portion of the additive particles will be present at outer and/or inner surfaces of the fiber walls, effectively imparting antimicrobial characteristics to the hollow fiber and any fibrous products made therefrom. The additives can be selectively dispersed in certain regions of the fibers in order to reduce the amount of the additives required, and are resistant to separation from the fiber wall, prolonging the fiber's antimicrobial effectiveness. Additional additives can be dispersed in the fiber wall with the antimicrobial agents in order to enhance or provide different fiber properties.

### CROSS-REFERENCE TO RELATED APPLICATIONS

This application is a continuation-in-part of co-pending U.S. application Ser. No. 10/762,920 filed 22 Jan. 2004, which is a divisional and continuation-in-part of U.S. Pat. No. 6,723,428, which claims the priority of the following provisional applications: Ser. No. 60/136,261 filed 27 May 1999; Ser. No. 60/173,207 filed 27 Dec. 1999; Ser. No. 60/172,285 filed 17 Dec. 1999; Ser. No. 60/172,533 filed 17 Dec. 1999; Ser. No. 60/180,536 filed 7 Feb. 2000; Ser. No. 60/181,251 filed 9 Feb. 2000; and Ser. No. 60/180,240 filed 4 Feb. 2000. All of said applications are incorporated herein by reference as though set out at length herein.

### FIELD OF THE INVENTION

The present invention relates generally to fibers, and, more particularly to pure hollow and mock-hollow fibers having anti-microbial (and/or anti-fungal) properties that are resistant to decreases in their effectiveness over time or repeated uses.

### BACKGROUND OF THE INVENTION

There is a growing demand in products which exhibit anti-microbial and anti-fungal properties. There are a number of additives, fibers and products on the market which claim to have these properties but do not, or the properties do not remain for the life of the product, or they have adverse environmental consequences.

Anti-microbial fibers may be used in a wide variety of fibrous products, among them textiles and garments (including athletic wear, incontinence and medical garments, etc.), air and water filters, wound and burn care dressings, medical wipes, shoe components, and institutional and home furnishings including bed sheets, pillow cases, mattress pads, blankets, towels, drapes, bedspreads, pillow shams, carpets, walk-off mats, napkins, linens, wall coverings, upholstered furniture, liners, mattress ticking, mattress filling, pillow filling, carpet pads, upholstery fabric and the like.

Hollow fibers have one or more continuous axially extending voids running through them. It is well known to use hollow and/or mock hollow fibers (hereinafter referred to collectively as “hollow fibers” unless otherwise specified) in many of the applications listed above, as well as in semi-permeable membranes for gas separation, blood dialysis, ultrafiltration, purification of water, and other filtering applications. When used in carpeting, hollow fibers require less fiber to produce carpet, thereby reducing manufacturing costs. For membrane applications, the hollow fibers can be bundled together and disposed in a tubular housing to provide separation devices known as permeators. Hollow fibers may also provide desirable thermal properties, as well as soil hiding ability, luster, and/or transparency. Yarns manufactured from hollow fibers are becoming increasingly popular in the synthetic fiber industry, due at least in part, to the improved performance and process efficiencies they represent. Each of these applications have various requirements including pore size, strength, biocompatibility, and/or production costs.

One of the disadvantages of some of the prior art is that anti-microbial additives are applied topically to the fibers or fabrics and tend to wash off or wear off over time and become ineffective. Also, by washing off the additives are placed into the waste water stream.

Thus, a need exists for anti-microbial (and/or anti-fungal) fibers that maintain their anti-microbial effectiveness even after repeated uses (e.g., washings, etc.) and which exhibit the additionally beneficial properties of hollow fibers.

### SUMMARY OF THE INVENTION

The present invention provides synthetic hollow or mock-hollow fibers exhibiting anti-microbial and/or antifungal properties. Such fibers can be composed of a variety of synthetic thermoplastic polymers having additives or combinations of additives, including anti-microbial (and antifungal) agents, selectively dispersed within the walls of the fibers. The type and concentrations of additives is controlled to achieve a hollow or mock-hollow fiber having the desired properties.

In another aspect, the present invention provides fibrous products manufactured from such hollow fibers, such as, for example, fabrics (woven and non-woven) comprised of the anti-microbial hollow fibers with or without blended synthetic or non-synthetic fibers possessing limited or no anti-microbial properties, filter materials and membranes, garments (including athletic wear, incontinence and medical garments, etc.), wound and burn care dressings, medical wipes, shoe components, and institutional and home furnishings including bed sheets, pillow cases, mattress pads, blankets, towels, drapes, bedspreads, pillow shams, carpets, walk-off mats, napkins, linens, wall coverings, upholstered furniture, liners, mattress ticking, mattress filling, pillow filling, carpet pads, upholstery fabric and the like.

The anti-microbial agents are efficacious and adhere to the fiber and are greatly resistant to washing off or wearing off of the fiber or fabric to which they are applied.

The anti-microbial additives utilized, alone or in combinations, are organic or inorganic. They may be applied to only selected fiber areas, or may be applied in higher concentrations in certain areas, to reduce the amount of the anti-microbial agent which needs to be used and thus lower the cost of such fiber and/or a fabric including such fiber.

One or more additional additives may optionally be dispersed with the agents in the hollow fiber material, including color pigments, UV additives, hydrophilic or hydrophobic additives, flame retarders and/or resistors, and/or anti-stain additives.

The anti-microbial and/or other agent(s) are held in the hollow fiber wall and are exposed externally by suitable sizing of particle cubes relative to the fiber wall thickness, e.g., a four micron cubic particle will have a maximum dimension of approximately 7 microns (1.73×4 micron) and be most effective in a fiber having an approximate thickness of 8 microns. More generally, the agent(s) particles size are chosen such that the fiber wall thickness will be in the range of 1.73 to 2.25 times the nominal size of the particle. These dimensions relate to the cross-sectional diameter of the cubic particles, the largest expected dimension of the particles. This relation between particle size and fiber wall thickness ensures that a portion of the agent particles will be externally exposed at the fiber wall, thereby imparting anti-microbial properties to the fiber.

Antimicrobial agents may be chosen from inorganic additives such as copper, silver, tin or zinc, incorporated in carriers such as zirconium phosphate, zeolites, or dissolvable glass. Organic agents may include triclosan and/or other antimicrobial chemicals.

The materials from which fibers may be produced include thermoplastic polymers such as polyester, nylon (polyamid), rayon, lyocell, polypropylene, polyethylene, aramid, acrylic, and the like.

As noted above, the anti-microbial finished products can be composed entirely of hollow fibers or blended with non-anti-microbial fibers such as cotton, wool, polyester, acrylic, nylon, and the like. PETG may be used as one of the polymer blends and/or carriers for a wide variety of applications. PETG is an amorphous binder fiber that can be blended into yarns with other fibers to form woven fabrics, as well as knits and non-woven fabrics. It is used in certain embodiments of the present invention as a carrier to carry pigments and/or anti-microbial additives and/or other additives and is blended with other fibers which may be natural fibers such as cotton, silk, flax, wool, etc. or other synthetic fibers such as: PET, PP, PE, Nylon, Acrylic, etc. After heat activation, the PETG melts, continuously releases the color pigments and/or anti-microbial or other additives and wets the surface of the surrounding fibers with the pigment and/or anti-microbial or other additives it carries. It settles at the crossing points of the fibers, thus forming “a drop of glue” which bonds the fibers together. Therefore, PETG delivers and distributes the pigments and/or anti-microbial or other additives uniformly within a fabric, generating the finished fabrics and/or fabrics having anti-microbial properties. Since the natural fibers used to blend with PETG are not changed physically after heat activation of PETG, they contain the same characteristics as natural fibers.

Anti-microbial fabrics may be made by blending hollow fibers composed, for example, of PETG (as a carrier for pigments and/or anti-microbial additives), with cotton or any other fibers of synthetic material such as from polyester and rayon, and activating PETG from 110° to 140° C. The color is thus provided to the yarn and fabric without the need of going through a dye bath. This fabric remains color-fast for in excess of 50 commercial launderings. The excellent wetting characteristics of PETG can be used to distribute the pigments and/or anti-microbial additive uniformly within a yarn or fabric. While many anti-microbial agents may be used, such as those, which use copper, zinc, or tin, the preferred agent is zeolite of silver. In addition to the anti-microbial component and the pigment added to the PETG, the PETG may be used as a carrier to add other properties to yarn and fabric, such as fire retardants.

The concentration of the anti-microbial agent (or combination or agents) can be varied within each individual hollow fiber as a gradient using selective mixing and extrusion strategies. The concentration of anti-microbial agent within such a fabric or material can also be varied regionally using hollow fibers containing varying amounts of anti-microbial agents in conjunction with both natural and synthetic fibers having different amounts of anti-microbial agents. A variety of other agents can be added, either by mixing or topically, for different reasons, such as altering its water absorbing qualities. Various polymers can be used to form these fibers. In the context of this invention, anti-microbial refers, but is not limited, to anti-bacterial and anti-fungal. In a preferred embodiment, a combination of copper and silver agents are dispersed in the hollow fiber wall.

### BRIEF DESCRIPTION OF THE FIGURES

Other objects, features and advantages will be apparent from the following detailed description of preferred embodiments taken in conjunction with the accompanying drawings in which:

FIGS. 1A-1D are perspective and cross-sectional views of various fiber configurations used in practice of the various embodiments of the invention;

FIG. 2 is a sketch of a fibrous mass using one or more of the fibers of FIGS. 1A-1D;

FIG. 3 is a schematic view of the feed hopper, screw and extruder;

FIG. 4 is a sectional view through the exit of the extruder showing the formation of hollow fibers of the present invention;

FIGS. 5 and 6 are photomicrographs of fibers showing the particles of zeolite of silver;

FIG. 7 shows a garment made from the fibers of the present invention for a person who is incontinent;

FIG. 8 is a cross section of one type of filter using the fibers of the present invention;

FIGS. 9A-9D are diagrams of air flow systems utilizing the fibers of the invention;

FIG. 10 is a cross section of one type of wound care or burn dressing;

FIG. 11 is a flow chart showing the preparation of the fibers and yarn for use in making a woven or nonwoven fabric;

FIG. 12 is a flow chart showing the preparation of fibers and yarn and then of a fabric;

FIG. 13 is a schematic isometric view of a first type of insole using latex;

FIG. 14 is a schematic isometric view of a second type of insole using a layer of anti-microbial fibers;

FIG. 15 is a cross-sectional exploded view through an office partition;

FIG. 16 is a schematic view of a humidifier evaporation surface media used to humidify air;

FIG. 17 is a schematic view of a humidifier pad or filter in a system;

FIG. 18 is an illustration of a circulation/aeration system including a filter in accordance with an embodiment of the invention;

FIG. 19 is a cross section through a laminate for footwear components;

FIG. 20 is a cross section through an insole made in accordance with the present invention; and

FIG. 21 is a plan view of the insole of FIG. 20.

### DETAILED DESCRIPTION OF THE INVENTION

In the United States, all claims concerning antimicrobial and antifungal properties must be thoroughly tested to Environmental Protection Agency (EPA) and Food and Drug Administration (FDA) standards. As used herein, the term “antimicrobial” refers to the ability of an article to kill 99.99% (log 3) of bacteria in 24 hours. The EPA has indicated that such products may be labeled as “Prohibiting Bacteria Growth and Migration Along the Surface of the Product.” The addition of the antimicrobial particles in accordance with the invention inhibits the growth of mold and mildew or odor-causing bacteria in the fibers. The hollow fibers retain their efficacy after simulated use conditions so that the antimicrobial efficacy is maintain throughout the life of the product.

The Fibers and the Additives

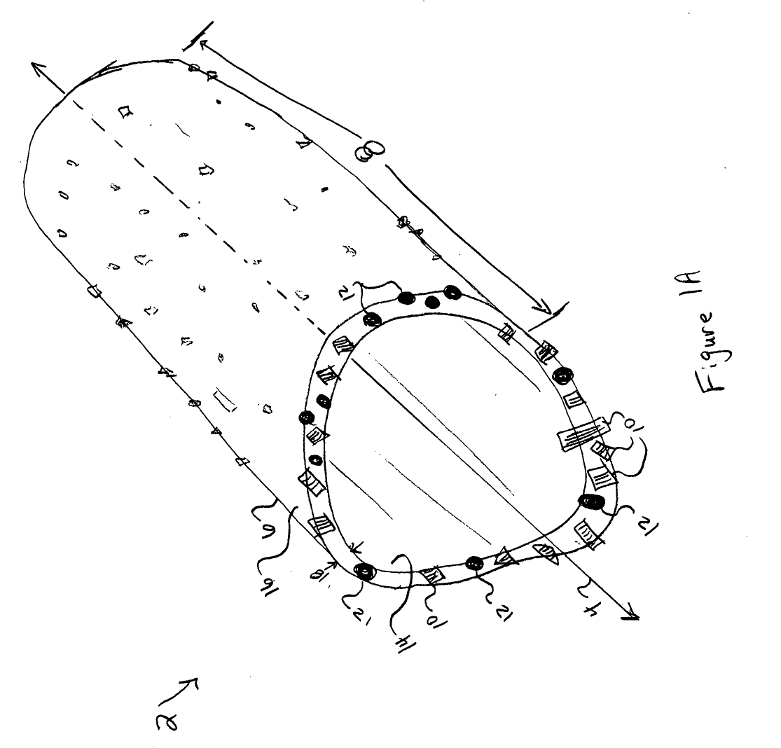

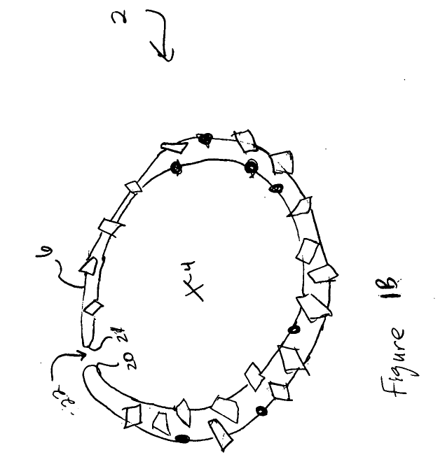

As the term “hollow” is used herein, it refers to fibers such as fiber 2 shown in FIG. 1 that has been formed through a pure hollow or mock-hollow fiber formation process or other suitable process. Fiber 2 includes an axially extending void (indicated by arrow 4) encompassed by a wall 6 extending the length 8 of the fiber. Organic or inorganic antimicrobial particles 10 are embedded within the wall 6 of the fiber as a result of the formation process, and are greatly resistant to separation from the fiber. Through mixing and extrusion strategies described below, the concentration of antimicrobial particles 10 in different portions of the hollow fiber may be controlled, reducing in some cases the amount of the antimicrobial agent required to manufacture an efficacious fiber and/or product incorporating such fibers.

The antimicrobial particles 10 and/or other additives 12 are interspersed in fixed positions in the wall 6 in such a way that a substantial number of the particles have some portion present at either or both the internal surface 14 or internal surface 16 of the fiber 2. The particles and/or additives are constrained to positions at or near the internal and/or external surfaces by suitable sizing. The particles 10 generally exhibit a cubic shape, and the nominal thickness 18 of the wall 6 formed so as to be from about 1.73 to 2.25 times the nominal size of the particles employed (i.e., roughly to coincide with the maximum cross-sectional diameter of a cubic particle). For example, a fiber 2 with an 8-micron wall 6 would obtain maximum effectiveness with a 4-micron cube (4×1.73 microns). Thinner wall thicknesses, however, may be employed. For example, the wall thickness could be roughly the same or even slightly less than the nominal dimension of the antimicrobial particle. Similar dimensioning may be applied for the additives 12 if present in a static particulate shape. In this manner, the antimicrobial particles 10 and additives 12 may be firmly embedded in the wall material. For larger or smaller particles 10, The thickness 18 of the wall 6 is selected according to the size of antimicrobial particles employed. As shown in FIG. 1A, antimicrobial properties may be imparted to both the external surface 16 and internal surface 14 of the fiber 2.

The wall 6 of the fiber 2 may be composed of various polymers, including but not limited to, polyester, nylon (polyamid), rayon, lyocell, polypropylene, aramid, acrylic, polyethylene (PE), polypropylene (PP), polyethylene terephthalate (PET), PCT, PETG, Co-PET and co-polyesters generally, Styrene, polytrimethylene terephalate (PTT), 3GT, Halar™, polyamide 6 or 6,6, and/or other thermoplastic polymers. PETG is an amorphous binder fiber material that can be blended into yarns with other fibers to form fabrics, as well as non-woven fabrics. After heat activation, the PETG fiber melts, wets the surface of the surrounding fibers, and settles at the crossing points of the fibers, thus forming “a drop of glue” which bonds the fibers together and distributes the antimicrobial additives.

The antimicrobial/antifungal particles 10 may comprise one or more inorganic particles such as copper, silver, tin, and zinc, that may be carried in compounds such as zirconium phosphate, zeolites, dissolvable glass or similar carriers. Alternatively, the antimicrobial/antifungal particles 10 may be triclosan and other known antimicrobial chemicals. High efficacy can be obtained using a zeolite of silver dispersed in a polyethylene (PE), PET, or polybutylene terephthalate (PBT) carrier, but the silver particles could be added directly to a melt of a wall thermoplastic without an intermediate carrier. Silver is the only suitable inorganic antimicrobial particle for some applications, as it is considered biocompatible with the human body. Combinations of antimicrobial particles, such as copper and silver, may also be employed to increase antifungal properties.

The hollow fiber 2 can be formed in a range of sizes (e.g., from 0.7 dTex to 25.0 dTex) and could be produced as a cut staple fiber in lengths from 1.0 mm to 180 mm, or in a continuous filament.

The additives 12 may be included in the fiber formation process in order to impart additional (non-antimicrobial) desired properties to the fiber and finished products incorporating the fibers. For example the additional additives 12 could comprise UV stabilizers, fire or flame retardant (FR) additives, pigments, hydrophilic or hydrophobic additives, anti-odor and/or anti-stain materials.

A cross-sectional view of second configuration of hollow fiber 2 is shown in FIG. 1B, wherein hollow fiber 2 was formed in a mock hollow fiber forming process incompletely joining two ends 20,21 of an extruded wall 6. This results in an axially extending discontinuity 22 in the wall 6 running the length of fiber 2. Mock hollow fiber formation may also result in a more complete joining of the two ends 20, 21 such that the ends flow together to seal the wall 6 of fiber 2 in a configuration such as depicted in FIG. 1A. The presence or absence of such a discontinuity 22 can be controlled during the mock hollow fiber forming process.

FIGS. 1C-1D illustrate other configuration of hollow antimicrobial fiber 2 wherein the antimicrobial particles 10 (and additives 12) have been selectively constrained to one or more sections of the fiber. The concentration of antimicrobial particles is controllable during fiber formation, and the concentration can be varied in axially extending portions of the wall 6 as shown, but could also be varied over the length of the fiber.

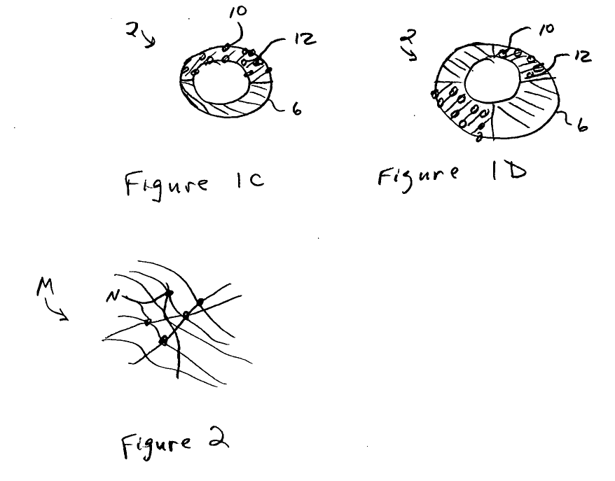

The hollow fiber may also serve as a binder or as part of a yarn or fabric with cooperating (strength) fibers. It should be understood that the nominal “binder” fiber or binder component can also be a strength enhancer in some combinations. FIG. 2 shows a non-woven or woven fibrous mass M made up of any of the fibrous configurations of FIGS. 1A-1D after heating, wherein the hollow binder fiber melts and flows to form locking knots at many (if not most or all) of the cross-over points or nodes N of the fibrous mass to enhance strength and durability of the mass while maintaining a dispersion of the binder materials and its functional additive(s).

While a preferred embodiment includes a wall composed of PET having zeolite of silver interspersed therein, resins with different viscosities can be used to obtain improved performance. A PCT wall takes advantage of the hydrolysis resistance and resilience of that polymer, however, PET is more cost effective, especially for use in apparel and bedding.

Referring again to FIG. 1A, the additives 12 may include pigments providing uniform colors that do not fade significantly over long-term use and washing, unlike dyes. Compounds may be used which create a hydrophilic surface designed to wick body moisture away from the skin and evaporate to create comfort for a wearer of a garment containing such fibers and is particularly useful for career apparel such as uniforms, work clothes, etc. The antimicrobial, anti-fungus and anti-odor additives can be varied depending on the functionality of the career apparel.

The hollow fiber could be produced from low temperature polymers with a melting or softening temperature below 225° C., such as PETG (PET modified with 1,4, cyclohexanedimthanol), PE, PP, co-PET, or amorphous PET. Another low melting temperature polymer which may be used is polycaprolactam (PCL). An effective compound could be a zeolite of silver dispersed in PE, PET, or PBT before being added to the fiber. The additives could be added directly to the primary polymer with pre-dispersion.

The binder (carrier) fiber composed of the polymer(s) and antimicrobial particles can be blended with non antimicrobial natural fibers such as cotton and wool, or synthetic fibers such as polyester, acrylic, nylon, PTT, 3GT, rayon, modified rayon, and acetate to form antimicrobial finished fabrics able to withstand significant wear and washings and maintain their effectiveness. A typical example is a hollow fiber using the PETG polymer with the zeolitic contained silver additive blended with cotton up to 10% by weight to produce a bed sheet. The binder fiber is activated in the drying cycle of the final bleaching operation or other heat operation. The PETG melts and wets the surface of the cotton fibers to carry the antimicrobial characteristics to the entire sheet with an added benefit of increasing strength and reducing pilling.

The antimicrobial end products can withstand more than 50 commercial washings and/or dry cleanings at 80° C. Such products are immune to UV exposure of at least 225 kj, and possess excellent abrasion resistance and are unaffected by tests such as Tabor or Wyzenbeek.

PETG has two characteristics of interest: (1) excellent wetting and (2) low melting temperature. In the present invention, it is used as a carrier to carry antimicrobial particles and is to be blended with non-antimicrobial fibers. After heat activation, the PETG melts, continuously releases the antimicrobial additives and wets the surface of the surrounding non antimicrobial fibers with the antimicrobial additives it carries. Thus, PETG delivers and distributes the antimicrobial additive uniformly within a fabric and the PETG holds the antimicrobial agent in place, generating the finished fabrics having antimicrobial property. Since the natural fibers used to blend with PETG are not changed physically in this process, they contain the same characteristics as natural fibers.

The hollow fiber may be formed by using pellets of multiple polymers or a direct polymer stream from the reactor of which the fiber is to be formed. The arrangement shown in FIG. 1A is intended for a configuration of a hollow fiber containing an additive, e.g., an antimicrobial agent. A production goal is to use as little antimicrobial material as necessary to provide the desired characteristics. The additive provides the desired antimicrobial effect only at the surface. If the bulk of the additive is located within the volume of the fiber wall not near the surface, that portion will not be useful for most or all of the life of the material into which the fiber is made. Since there frequently is some surface abrasion, some of the additive particles which are just below the surface when the fiber is made, become available at the surface, later in the life of the product.

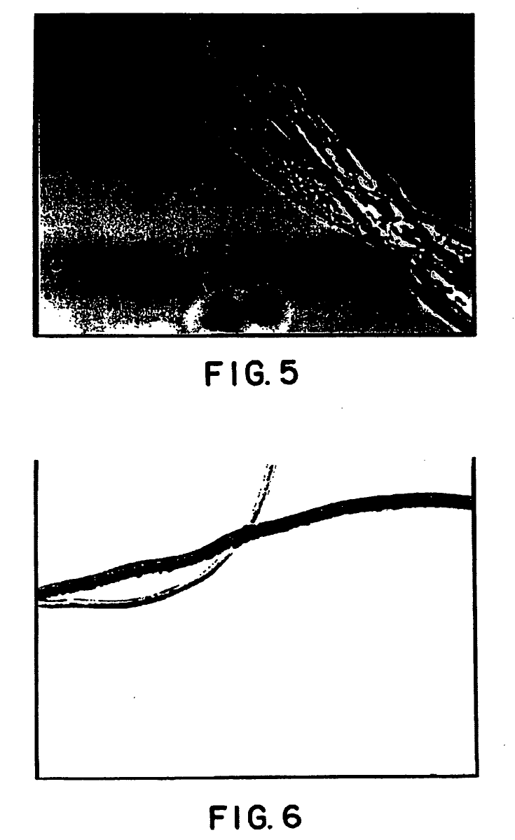

It has been possible to make particles of zeolite of silver as small as 1 μm cubes. A particle of such size will have a diagonal dimension of about 1.7 μm. Therefore, the smallest thickness of the fiber wall 6 should be about 2 μm. The present invention permits an arrangement in which the wall is as small as 2 μm in thickness. In preferred arrangements, most or all of the additive is available for surface (internal and/or external) action. With significant wear of the surface of the fiber, other additive particles which were originally more deeply embedded, become available at the surface. The photomicrographs of FIGS. 5 and 6 of the non-hollow fibers of the parent application show small particles of zeolite of silver, many of which can be seen on the surface or projecting through to the surface of the fibers. There are more such particles which are just below the surface of the fibers, and which will become available for antimicrobial activity as small portions of the fiber wears or washes away and the particles become available at the surface.

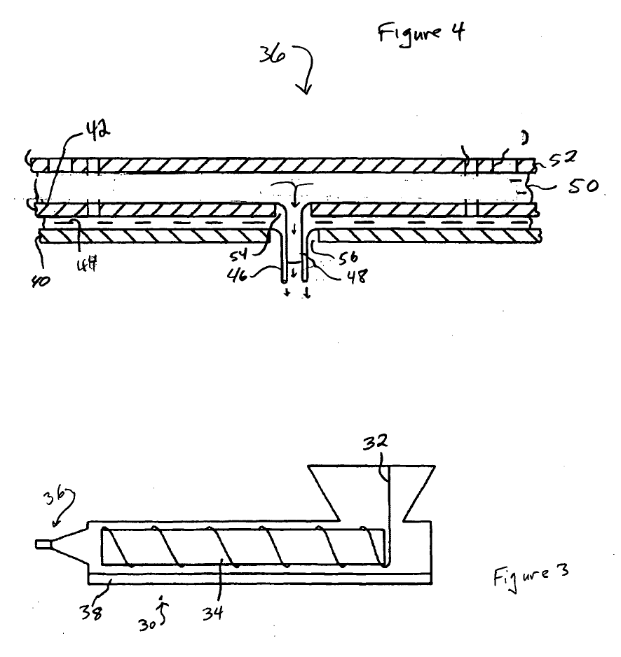

FIGS. 3 and 4 illustrate one approach of making a hollow antimicrobial fiber. The extruder 30 is shown diagrammatically in FIG. 3 having a feed hopper 32, an extruder screw section 34 for feeding melted material to the delivery end 36, and a heating chamber 38 which surrounds the bottom of the feed hopper as well as the total length of the extruder screw section 34 that melts the polymer (pellets) fed into through hopper with a controlled amount of antimicrobial particles and or other additives, and maintaining the polymers in molten condition until extrusion through nozzle openings at end 36. It is also possible to make the fibers using direct polymer streams from continuous reactors feeding to melt pumps (not shown.)

The nozzle end 36 of extruder 30 is shown in cross section in FIG. 4, which includes two sheets of metal 40 and 42 forming a molten polymer chamber 44. The melted polymer mixture is fed into the extruder die from the top A fluid source (e.g., air) from fluid chamber 50, which is formed between plate 42 and plate 52, flows through a mandrel or capillary 48 passing substantially concentrically through an apertures 54, 56 in plates 42 and 40, respectively. The molten polymer/additive mixture is guided in an annular distribution along the capillary 48 as it extrudes from chamber 44 through aperture 56 in plate 40. The positions of plates may be retained by means known in the art, such as by a central housing (not shown) or by screws or bolts threaded into the plates (not shown.) Capillary 48 facilitates formation of the axially extending void in the extruded hollow fiber. Capillary 48 terminates at an opening through which the fluid flowing through the capillary exits. The flow of fluid through the now-formed hollow fiber helps prevent closure of the void between the walls of the fiber and may assist in cooling the polymer material. All relevant apertures shapes, diameters, relative component separation distances, flow rates, etc., are selected based upon the geometry (size and shape) of the hollow fiber desired. The use of liquid as the void/core fluid may be used instead of air or another gas.

Other hollow fiber forming systems utilizing conventional spinnerette assemblies and/or solution precipitating processes may also be employed.

Also, the antimicrobial fibers may be formed by mock hollow forming processes wherein, generally, a thin film of finite width is extruded and folded while still in a near molten state into an annular cross sectional geometry so that the ends of the film contact each other and thereby flow together forming a hollow fiber. There may be instances wherein it is desirable to have the ends come into close proximity but not flow together, thereby forming a fiber with an axially extending discontinuity in the fiber wall, such as shown in FIG. 1B.

A wide number of end products may be formed from hollow antimicrobial fibers. In co-pending application U.S. Ser. No. 10/762,920, the contents of which are incorporated herein by reference, the inventor describes a wide variety of end products that may be produced with antimicrobial fibers generally. All the end products described therein may also be produced using the hollow antimicrobial fibers of the present invention, and can equally be expected to produce end products having a 99.99% microbial kill ratio.

The antimicrobial hollow fibers described above can be used to make both woven and nonwoven fabrics as well as knitted fabrics. Such fabrics are useful for various types of articles, some of which are listed below.

Incontinence Garments

Incontinence garments include disposable diapers, underwear, pajamas, and linens, some of which may be knitted. Such garments and other articles for incontinent persons may be produce from antimicrobial fibers composed of various thermoplastic polymers and additives. The antimicrobial fibers could be distributed only in certain areas of the garment in order to reduce the amount of the antimicrobial agents being used, and therefore the cost of such fibers. The antimicrobial additives used in the synthetic fibers do not wash off over time because they are integrally incorporated into these fibers, thus their effectiveness is increased and prolonged. The antimicrobial synthetic hollow fibers may comprise high tenacity polymers in one portion and/or hydrolysis resistance polymers (e.g. PCT) in another component. The hydrophilic and antimicrobial additives provide a hydrolysis-resistant surface with good wrinkle resistance that results in long-term protection against washings in boiling water and strong soaps. The antimicrobial synthetic fibers can further be blended with non-antimicrobial fibers such as cotton, wool, polyester, acrylic, nylon, etc. to provide antimicrobial finished fabrics that are able to withstand significant wear and washings and maintain their effectiveness. Antimicrobial fibers can be used to make materials for a variety of applications in which it is necessary or desirable to reduce bacterial and fungal growth and the resultant odor. Specifically, in personal hygiene situations, these materials can be used in reusable or re-wearable incontinent garments and other articles such as linens and bed packs to prevent bed sores on persons confined to bed for extended periods of time. Diapers and other clothing and articles for incontinent individuals are constantly and intermittently being soaked with urine and these items as now manufactured are not effective at killing odor and infection-causing bacteria. By making these items disposable, the growth of bacteria and fungi is reduced depending upon how often they are changed, but there are environmental and other considerations to disposables. However, the use of the antimicrobial fibers in such garments and articles that maintain their effectiveness during washings, results in reusable garments and articles of the type described with odor reducing and antimicrobial properties which last for the life of such garments and articles.

Antimicrobial fiber-containing garments are useful in reducing the growth of bacteria, fungi, and other microbes once soaked with urine, thus reducing the discomfort of the individual and preventing infections generally. Specifically, the antimicrobial fiber-containing fabrics may be used in both the covering fabric and the water absorbent interior material. In this way, both surface and interior protection is achieved. In addition, these materials may be reused. Thus, a significant cost savings is realized in the laundry operations of hospitals and nursing homes as well as in the economics of individual households.

The strength and resiliency of these garments is important since they must stand up to multiple wettings and subsequent cleanings. Thus, both hollow fibers and mixed fiber fabrics may be useful in incontinence garments. Also, other modifications of the characteristics of these fibers and fabrics beyond that of adding antimicrobial agents, including the addition of agents to increase or decrease hydrophobicity may be useful. In addition, anti-odor additives may be particularly useful in this application in light of this frequency of cleaning, as well as the wetting with urine. Thus, these antimicrobial materials, garments and articles significantly reduce the growth of mold, mildew, and bacteria in home and institutional environments.

Garments for incontinent persons may be composed of antimicrobial fibers incorporating inorganic silver-containing compounds, however, other metals (such as copper, potassium, magnesium, and calcium) can be used as antimicrobial agents. In addition, mixtures of different metal-containing antimicrobial agents in differing concentrations can be used that result in hybrid agents tailored for specific tasks.

Such garments may be knitted or woven and include underwear, pajamas, linens, disposable diapers, and the like.

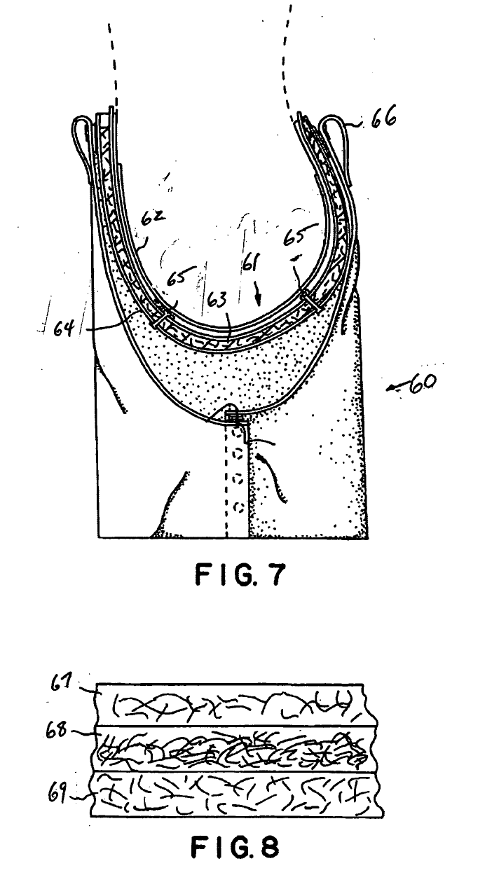

FIG. 7 shows a garment 60 having a removable liner assembly 61. The liner assembly includes an outer layer 62 that contacts the skin of a wearer. This layer is made to be smooth and soft so as to be comfortable for the wearer even when fluids such as urine contact this layer and pass through. There is a wick layer 63 that changes color when wet so that attendants can see from a distance that a wearer is wet and needs to receive some attention, such as the changing of the liner assembly. Beyond the layer 63 is an absorbent layer formed of a mass of fibers. There is an inner layer 64 which is impervious to fluids so that the fluids such as urine do not wet and/or stain the outer layer of clothing. The liner assembly 61 is held together by soft fiber connectors 65. The liner itself may be removably attached to the basic garment with Velcro so that it is easily removable and changed. The liners 61 may be constructed to be washable for reuse, or disposable. The garment has a belt 66 for holding the garment in place. The outer layer 62 may be made of the antimicrobial hollow fibers for protection from microbes and fungus which causes infection and odors.

Layer 62 may be porous in order to wick moisture away from the wearer and into the absorbent liner. Since the layer 62 contacts the wearer's skin and may at times be wet, there is the risk of infection that is reduce by the antimicrobial fibers.

The absorbent material of the liner 61 may also be made of non-woven fibrous material incorporating antimicrobial hollow fibers if desired. Antimicrobial fibers may be made into other products intended for incontinent persons, such as bed linens, and bed packs which are used to prevent bed sores in persons who are confined to bed for extended periods of time. Such products provide a first line of attack against problems caused by microbes especially when used in all areas of the products which come into contact with a person's skin.

Higher loading of the antimicrobial agents (up to 5 times) and/or combinations of antimicrobial agents maybe used to more effectively act against fungi. This higher loading may be achieved by using various zeolites followed by heating the fiber polymer, e.g. PET, to between 180 and 230° F. in hot water, which allows further metal loading or ion exchange to replace resident metal ions with another ion or mixture of ions. In addition, this would allow the zeolite at or near the surface of the fiber to be preferentially loaded with the metal ion or mixtures thereof that has the desired biological effect. These methods are particularly useful in reducing costs when expensive metal ions, such as silver, are used in these processes. Also, by adding certain metals, e.g. silver, at this point in the process and not having it present during the high temperature fiber extrusion process, any yellowing or discoloration due to oxidation of the metal ion or its exposure to sulfur and halogens would be greatly reduced.

Filters

Air filters for HVAC systems, air conditioning systems, car and airplane cabin systems, and ultrafiltration systems for fluids may include the antimicrobial hollow fibers, which can reduce bacterial and fungal growth and the resultant odor. Specifically, in vehicles, such as automobiles, the air filters and attached air conditioning units are the source of musty smells associated with the seeding and growth of bacteria, fungi, mold, and mildew. Because of the recirculation of outside and air-conditioned air through these filters, very favorable conditions exist for the growth of bacteria, fungi, and other microbes. Also in aircraft cabins, the air filters have the same beneficial results. The concentration of the antimicrobial agents may be varied within each individual fiber as a gradient using mixing strategies and also from fiber to fiber. The concentration of antimicrobial agent within a fabric or material made from these antimicrobial fibers can also be varied regionally using fibers containing varying amounts of antimicrobial agents in conjunction with both natural and synthetic fibers having different amounts of antimicrobial agents or even no added antimicrobial agents. A variety of other agents can be added, either by mixing or topically, to color the fibers and/or to make it resistant to staining, fire, and ultraviolet (UV) light as well as altering its water absorbing qualities.

Air conditioners are a source of musty smells associated with the seeding and growth of bacteria, fungi, mold, and mildew on the evaporator and or heater cores and housings. These areas, by their nature, collect dust, dirt, bacteria, mold spores, etc. in an environment that contains the moisture, temperature, and shielding from direct sunlight necessary to promote growth of these organisms. A filter containing permanent antimicrobial fibers, described herein, could be placed in the outside make-up air and/or recirculated air streams to kill the spores and cells trapped by the filter. This would reduce or eliminate the odors associated with growing and reproducing organism.

The permanent nature of the antimicrobial fibers in the filter is necessary based on the environment of operation and desired replacement life. The filters are subjected to moisture from entrained water from the blower fan inlet (rain, or wash water) as well as condensation of moisture when the air conditioning system is in operation. Further, the vehicle owners, and vehicle design engineers, want a filter that has at least a one year life. Both conditions can be overcome with permanently antimicrobial fibers described herein.

Such antimicrobial fiber-containing filters are useful in reducing the build-up of biological materials and films on the filters themselves and the associated air conditioning units. Thus, they would also be less likely to impart undesirable odors to the interior of the vehicles.

In manufacturing these materials, any of the embodiments described above could be used. Both the strength and resiliency of these materials is important given that they are used in continuously circulating air streams and are subject to the pressures characteristic of filtering processes. Any number of filter shape designs could be used as appropriate. In some instances, round filters would be appropriate whereas in other instances pleated or other shape filters would be appropriate, all depending on the pressure, volume characteristics of the air flow and available space. Both pure hollow fiber filters and blended fibers filters may be useful. FIG. 8 illustrates a three layer filter including a support layer 69, a filtration layer 68 made with antimicrobial fibers and then a pre-filter layer 67 also made with antimicrobial fibers.

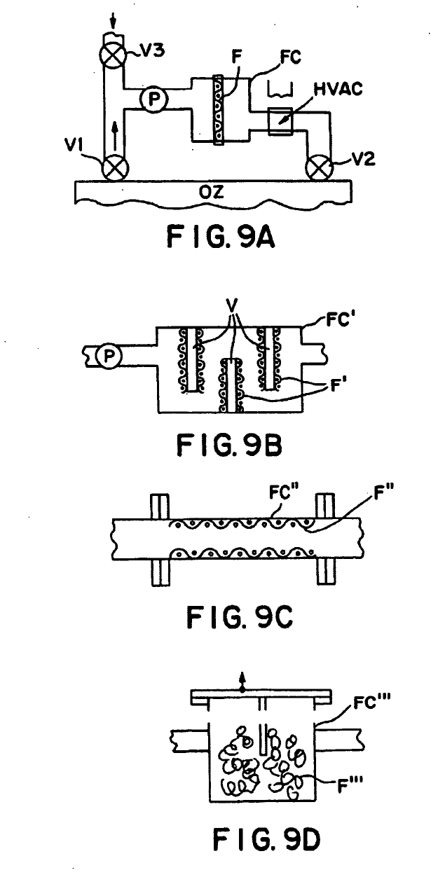

FIG. 9A shows a system of filter usage for an occupancy zone where air is removed via valve V1 through a pump or compressor P passed through a filter canister F (or other container) and a heating or cooling exchanger (HVAC) and returned to the occupancy zone via valve V2. The system can also handle outside air via a valve V3. The canister has a removable antimicrobial filter screen F (with a frame, not shown) removable for exchange or regeneration of antimicrobial effectiveness from time to time.

Another form of filter is shown in FIG. 9B as filter canister FC′ with vanes V defining a tortuous path, the vanes being lined with antimicrobial screening material F′.

FIG. 9C shows another form of canister as a tube FC″ lined with such filter material F″ and FIG. 9D shows a canister FC′″ with a loose array of filter material F′″ (similar to a scouring pad).

Wound Care Dressings and Burn Dressings

Wound care and burn dressings can incorporate the hollow fibers disclosed. The antimicrobial synthetic fibers can further be blended with non-antimicrobial fibers such as cotton, wool, polyester, acrylic, nylon etc. to provide antimicrobial finished wound care dressings and burn dressings that are able to withstand significant wear and any washings they may be given (if the washable type) and while maintaining their effectiveness.

Wound care dressings may be made with antimicrobial fibers used to make various materials for a variety of applications in which it is necessary or desirable to reduce bacterial and fungal growth. Because these dressings must be frequently changed and the wound exposed to pathogens during this changing process, the addition of antimicrobial agents to the wound care dressing helps to reduce the growth of these pathogens.

As a result of the above, the use of antimicrobial fibers in the manufacture of wound care dressings provides a practical medical article. These antimicrobial fiber-containing dressings are useful in reducing the growth of bacteria, fungi, and other microbes that can be introduced from the environment during the changing of dressings and while performing other manipulations, thus reducing and preventing infections generally. Specifically, the antimicrobial-fiber containing fabrics could be used in both the covering fabric and the water absorbent interior material. In this way, both surface and interior protection could be achieved. In addition, these materials could, if desired, be made to be reusable because the antimicrobial effect of the fibers of this invention are resistant to multiple washings. Thus, a significant cost savings could be realized in the purchasing of supplies in hospitals and nursing homes as well as in the economics of individual households.

In manufacturing these materials, any of the embodiments of fibers described above could be used. Both the strength and resiliency of these materials is important in that they must withstand normal patient movement and manipulation by health care workers. In addition, anti-odor additives may be useful in this application given the exposure of the dressing to various tissue exudates. Thus, these antimicrobial materials would then significantly reduce the growth of mold, mildew, and bacteria in wound care dressings.

Burn dressings may be made with antimicrobial fibers to make various materials for a variety of applications in which it is necessary or desirable to reduce bacterial and fungal growth. Because these dressings must be frequently changed and the burn exposed to pathogens during this changing process, the addition of antimicrobial agents to the burn dressing would help to reduce the growth of these pathogens.

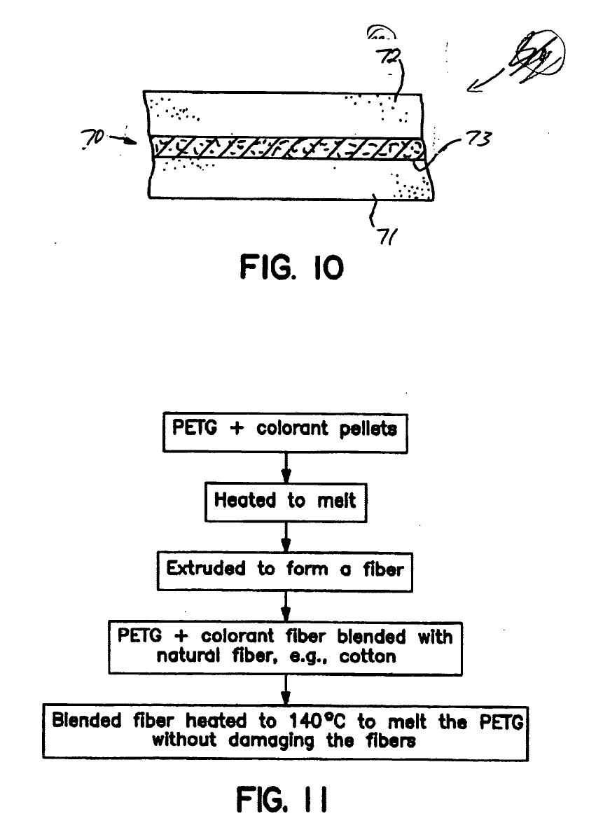

FIG. 10 shows a wound care or burn dressing 70 which includes a bottom layer 71, a top layer 72 and an intermediate absorbent fibrous layer 73 which joins the other two layers. The bottom layer 71 is used directly against the wound or burn and therefore the fibers of this layer have the antimicrobial agent applied thereto as described below. Both the strength and resiliency of these materials is important given that they must withstand normal patient movement and manipulation by health care workers.

After heat activation, the PETG fiber melts, wets the surface of the surrounding fibers, and settles at the crossing points of the fibers, thus forming “a drop of glue” which bonds the fibers together. PETG is also used to carry pigments and/or antimicrobial additives to the fibers, distribute the pigment and/or antimicrobial additives on the surface of the surrounding fibers, and achieve certain colors without the need to dye the fibers and natural fabrics having antimicrobial qualities. This invention presents a method for making a pastel shade fabric and/or nature fabrics having antimicrobial activities by using PETG as a carrier for pigments and antimicrobial additives, blending them with cotton or any other fibers, activating and melting PETG from 110° C. to 140° C., and leaving the encapsulated pigment and antimicrobial additives on the fibers. The final pastel shade fabric having an excellent fastness for both sunlight resistance and washing without the need of going through a dye bath, and the color may remain fast for a high number of commercial launderings. If the pastel shade fabric is made by blending PETG and pigments with cotton, after the activation of PETG, the final product can still be labeled as 100% cotton fibers. Thus, the present invention provides a fiber, yarn and/or fabric construction. There is a method for making a fiber blend which includes mixing a polyester polymer, characterized by a low melting temperature and having binder qualities, with an additive for providing desired characteristics to a finished fiber. The mixture is heated and extruded to form a continuous filament. The continuous filament fiber is cut to form a cut filament fiber. The cut filament fiber is blended with a natural fiber to form a fiber blend. The fiber blend is heated to a temperature in the melting temperature range of said polyester polymer for a sufficient period of time to melt the low melting temperature polyester polymer and wet the natural fiber and provide such natural fiber with the additive firmly attached thereto. The polyester polymer may be PETG. After the fiber is prepared it may be spun to make a yarn and the yarn may be made into a fabric. The heating step can take place after the yarn is made into a fabric. The additive may be a colorant, an antimicrobial agent, a fire retarding agent, or another agent which adds properties to the fiber or yarn or fabric. There is another method for making a fiber, which includes mixing a polyester polymer, characterized by a low melting temperature and having binder qualities, with an additive for providing desired characteristics to a finished fiber, heating the mixture and extruding it to form a continuous filament. Another polymer is heated and extruded to form a continuous filament. The extruding steps form an antimicrobial hollow fiber.

The fiber may be heated to a temperature in the melting temperature range of the polymer for a sufficient period of time to melt the polymer and wet higher temperature fibers near the hollow fiber, so as to firmly attached the additive(s) to the other non-hollow fibers. Additives can effectively be delivered to fabrics of fiber blends, using polymers such as PETG to carry and deliver pigments and/or antimicrobial or other additives to a natural fiber, such as cotton, wool, and the like, and generate a final pastel shade fabric without losing the natural fiber's characteristics and/or natural fabric having antimicrobial properties. PETG may be used as a carrier for pigments, such as carbon black, phthalo blue, and the like. Hollow PETG fibers may be mixed with other fibers, such as natural fibers, to form a blend, and then the blend is heated, to a temperature of around 140° C. (the PETG can be modified to melt between 90° C. and 160° C.) either as a separate heating step or during a processing step which includes heating to about temperature. PETG has a melting temperature of around 140° C. (and is available with melt temperatures from 90° C. to 160° C.) and it melts and flows along the fibers with which it is blended. It acts as a binder-carrier in that it forms nodes of color (when a colorant is used) with many points so it looks like a solid color. This provides it with a pastel look. By controlling the amount of colorant added to the PETG there is controllable color values which include pastel shading. PETG has superior wetting ability and therefore it spreads evenly along the other fibers with which it is blended. There are also nodes formed at the intersecting fibers in the blend and there are held together by this characteristic of the PETG. Also, the amount of PETG can be controlled to be small quantities with respect to the other fibers in the blend. Thus, when blended with cotton in this manner, such a blend may properly be characterized as “all cotton” having color and/or antimicrobial (or other) agents, which have been added by the PETG.

This can be accomplished in more than one manner. One method is shown in FIG. 11 in which the PETG and colorant pellets are mixed together, after which they are heated to melt and are then extruded to form a hollow PETG fiber with the colorant therein. The PETG is then blended with a natural fiber, such as cotton, to form a blend, which will have the color of the colorant, which the PETG fiber takes on as its color. The cotton is white so that the color taken on is a pastel color. If the colorant is black, then the blend becomes a shade of gray. If desired other fibers can be blended with the PETG fibers, such as silk, flax, polypropylene, polyethylene, wool, polyester, acrylic, nylon, PTT, 3GT, rayon, modified rayon, and acetate.

The PETG is then heated to a melt temperature that does not harm the fibers with which it has been blended. The PETG carrier melts and wicks along the other fibers, that is the cotton or other base fibers, forming small nodes, but it does not ball up as some polymers do and provides “a drop of glue” (small) to bind the fibers together and leaves behind the encapsulated pigment in the fibers. This fiber blend may then be used to form a yarn with in turn is used to form a fabric. The resulting fabric is a pastel shade fabric without the need of going through a dye bath, and has excellent color fastness from both sunlight and washing. The color is a pastel since there are many tiny drops of the colorant which looks like a solid color to an observer. The color remains fast for in excess of 100 commercial launderings. Since the PETG carrier melted after activation, the blended fibers such as cotton are still considered to be 100% cotton fiber.

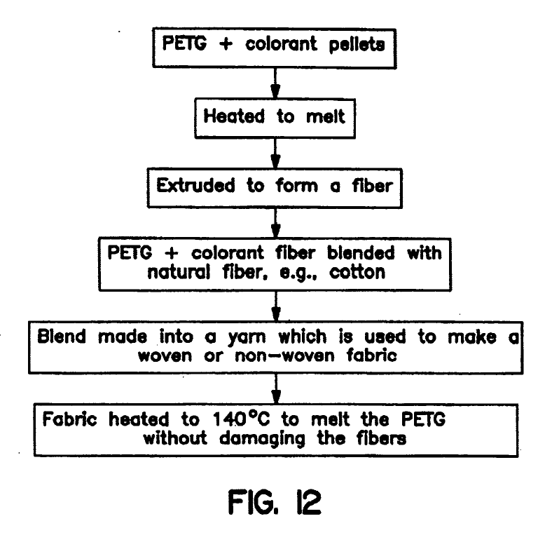

FIG. 12 shows a method similar to that shown in FIG. 11, however, in this process the blended fiber is made into a yarn and the yarn is made into a fabric before the PETG is activated by heating. This heating may be a separate heating step or may take place during the processing of the fabric which may include a heating step for other reasons.

The present invention may also be used to provide antimicrobial fibers by using PETG as a carrier for antimicrobial additives. Again the PETG and the antimicrobial pellets may be melted together to form a melt which is extruded to create a continuous filament which is then cut to appropriate size and is then further blended with natural or other fibers to provide an antimicrobial finished yarn which may be made into an antimicrobial fabric that is able to withstand significant wear and washings and maintain their effectiveness. The antimicrobial additives are inorganic compounds made from metals such as copper, tin, zinc, silver, and the like. The preferred compound is a zeolite of silver which may be dispersed in PE, PET, or PBT before being added to the fiber. The additives can be added directly to the primary polymer with pre-dispersion. The total active ingredients range from 0.1 to 20% by fiber weight. Other inorganic metals such as tin, copper and zinc work also, but not as well as zeolite of silver.

The PETG polymers with antimicrobial additives can be blended with natural fibers such as cotton, silk, flax, and wool, or synthetic fibers such as polyester, polypropylene, polyethylene, acrylic, nylon, PTT, 3GT, rayon, modified rayon, and acetate to make antimicrobial finished fabrics that are able to withstand significant wear and washings and maintain their effectiveness.

A typical example is a fiber using the PETG polymer with the zeolite contained silver additive blended with cotton up to 10% by weight to produce a bed sheet. The binder fiber is activated during the drying cycle of the final bleaching operation or other heat operation. The PETG melts and wets the surface of the cotton fibers to carry the antimicrobial characteristics to the entire sheet with an added benefit of increasing strength and reducing pilling.

The fiber size ranges from 0.7 dTex to 25 dTex and a staple length of 1.0 mm to 180 mm. A continuous filament yarn can also be produced that can be used in a wrap spun application whereby fibers are spun around the antimicrobial filament. The antimicrobial product is expected to withstand more than 50 commercial washings at 80° C., and be immune to UV exposure of at least 225 kj. It will possess excellent abrasion resistance and is unaffected by tests such as Tabor or Wyzenbeek.

Footwear

Footwear components such as insoles, midsoles, box toes, counter and linings of footwear products, e.g., shoes, slippers, sneakers and the like are provided in which the antimicrobial agent is available for the life of the product and not washed away or worn away by sweat or abrasion. Also, the antimicrobial agent is placed into the component close to or on the surface which is most needy of the protection, such as the part of an insole closest to the foot of a user when the insole, or other component is assembled into a footwear product. Thus, the fungi or microbes which may form and create odors or other problems are killed on contact with the surface of the shoe component antimicrobial surface area. The footwear components can be a nonwoven fabric of synthetic fibers, primarily polyester, but which could be acrylic, nylon, rayon, acetate, PP, and the like. The fabric can have a weight from 65-400 grams per square meter and typical fibers range from 1.2 dTex to 7 dTex with a cut length of 25-76 mm. They are carded, cross-lapped and needle punched, but could be produced on other types of nonwoven equipment, such as spun laced or spun bonded equipment. The impregnation is a latex of SBR, vinyl acetate, PVC, acrylonitrile, and the like. Impregnation is from 1-4 times the weight of the nonwoven fabric on a dry basis. A range of fillers such as clay, calcium carbonate, and the like are used to reduce the cost. There are two basic methods. One is to mix the antimicrobial with latex compound and impregnate it into the insole. The other is to use antimicrobial fibers on the insole in various manners; The footwear components are provided by several embodiments described herein but may be practiced using other embodiments. There is described below, a first embodiment of a single layer of latex, and a second embodiment of a main support layer and a fiber layer attached thereto.

The footwear component can be a nonwoven fabric of synthetic fibers, primarily polyester, but which could be acrylic, nylon, rayon, acetate, PP, and the like. The fabric can have a weight from 65-400 grams per square meter and typical fibers range from 1.2 dTex to 17 dTEx with a cut length of 15-180 mm. They are carded, cross-lapped and needle punched, but could be produced on other types of nonwoven equipment, such as spun laced or spun bonded equipment.

The impregnation is a latex of SBR, vinyl acetate, PVC, acrylonitrile, and the like. Impregnation is from 1-4 times the weight of the nonwoven fabric on a dry basis. A range of fillers such as clay, calcium carbonate, and the like are used to reduce the cost. There are two basic methods. One is to mix the antimicrobial with latex compound and impregnate it into the insole. The other is to use antimicrobial fibers on the insole in various manners.

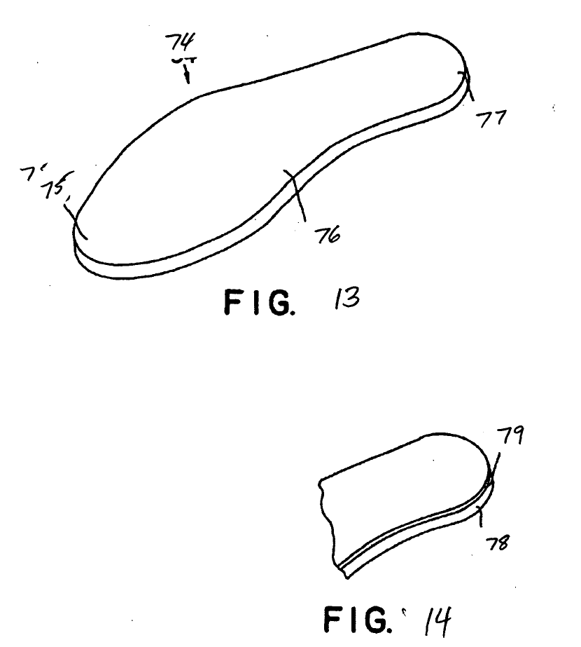

An embodiment of a nonwoven fabric impregnated with latex is shown in FIG. 13, which illustrates an insole 74 having a toe portion 75 and a mid sole portion 76 and a heel portion 77 all in a single piece construction. It is a suitable fabric which is then impregnated with latex to provide cushioning for wearer comfort. The antimicrobial, in this case zeolite of silver is mixed with the latex prior to impregnating the insole.

FIG. 14 presents another arrangement wherein a support and cushioning layer 78 is provided and which may be any of a number of materials which are used for insoles, but preferably one which of a nonwoven material. A fiber layer 79 made of fibers which have the antimicrobial agent disposed therein is attached to cushioning and support layer 78 by any suitable means. In this arrangement zeolite of silver is the antimicrobial agent. This can include an adhesive, but could also be accomplished by making the support layer of a polymer which is also used for some of the fibers and the fiber layer 79 is attached to the support layer 78 as the support layer is first delivered after being prepared and still retains the heat of preparation whereby the common polymer is hot enough to partially melt and then become bonded together.

Some antimicrobial agents (e.g., copper) are also anti-fungal agents. When agents do not perform both functions, a second agent may be used. The choice of particle size of the zeolite is based on the thickness of the layer carrying it to obtain the best combination of surface area with anchoring in the layer. For example, a very thin layer of 3 μm would be best served with a 1-μm zeolite, which would have a maximum dimension of 2×1.73 or about 3.5 μm.

The inner layer(s) could be made of basically any thermoplastic resin, such as; PE, PP, PET, PS, PCT, Polyamide (nylon), Acrylic, PVC, etc. The surface layer(s) could be made of the same polymers plus some low temperature ones such as PETG, Polycaprolactone, EVA, etc. It is preferable to have the layer closest to a wearer's foot have the antimicrobial and/or anti-fungal agent and be porous to perspiration to absorb perspiration. In the event a support layer is used which is not fibrous, it is covered with a nonwoven fabric, the fibers of which have the antimicrobial agent therein. Such a layer can be thinner than the support layer. However, it is usually best if the layers used allow perspiration to be carried away from the wearer's foot for both comfort and health reasons.

The antimicrobial particles are bonded into the surface layer and remain there for the life of the material and provide antimicrobial properties for the entire time. It is advantageous to have the antimicrobial agent only at the surface since this is the only area which comes into contact with microbes and fungi, and to have the agent located in other places is wasteful.

Antimicrobial fibers can be used to make the footwear products of the present invention where it is necessary or desirable to reduce bacterial and fungal growth and their resultant odor. In manufacturing these materials, any of the embodiments of fiber described can be used. Both the strength and resiliency of these materials is important. Any number of shaped designs could be used as appropriate.

Also, other modifications of the characteristics of these fibers and material beyond that of adding antimicrobial agents, including the addition of agents to increase or decrease hydrophobicity, would be useful. In addition, anti-odor additives may be particularly useful. The relatively small size of the silver-containing zeolite compounds (2 microns and less) that are used in the manufacturing of the fibers allow these antimicrobial agents to be incorporated into fibers instead of being applied to them. Thus, because these antimicrobial agents are an integral part of the fiber, they are not washed away by perspiration or easily abraded away and the finished components, such as insoles, manufactured from them are able to withstand significant wear while maintaining their antimicrobial effectiveness.

Specifically, higher loading of the antimicrobial agents (up to 5 times) is used to more effectively act against fungi. This higher loading may be achieved by using various zeolites followed by heating the fiber polymer, e.g. PET, to between 180° and 230° Fahrenheit in hot water which allows further metal loading or ion exchange to replace resident metal ions with another ion or mixture of ions. In addition, this would allow the zeolite at or near the surface of the fiber to be preferentially loaded with the metal ion or mixtures thereof that has the desired biological effect. These methods are particularly useful in reducing costs when expensive metal ions, such as silver, are used in these processes. Also, by adding certain metals, e.g. silver, at this point in the process and not having it present during the high temperature fiber extrusion process, any yellowing or discoloration due to oxidation of the metal ion or its exposure to sulfur and halogens would be greatly reduced.

It is also possible to use these integrated antimicrobial compounds to make shoe components and products that have a varying distribution of the antimicrobial agent. For example, by varying the concentrations of the antimicrobial agent during mixture with the fiber-forming polymers, fibers having varying antimicrobial content can be formed which can then be added in varying amounts to form materials having varying concentrations of antimicrobial agents. In addition, the amount of antimicrobial present in the fiber itself can be varied, either lengthwise or in cross-section. Similarly, higher and lower concentrations of these antimicrobial agents in the overall fibers can be achieved by using multi-layered sheets in which, for example, the antimicrobial agent is present only in an outer layer section, thus significantly reducing manufacturing and selling costs. Any of the above manufactured antimicrobial fibers can be mixed with fibers that do not contain antimicrobial agents such that products can be made having overall and localized variations in concentrations of antimicrobial agents.

In addition, the fibers can be made either hydrophilic or hydrophobic as desired by mixing other agents into the fiber polymers or applying them to the fiber surface. By modifying the wetability characteristics of the fibers, they can be made more useful for various applications. For example, hydrophilic fibers are effective in applications in which one wants the antimicrobial material to more easily absorb water, such as when the material is designed to be used in footwear. Alternatively, hydrophobic films or fibers are effective in applications in which one wants to avoid the absorption of such solutions. For example, the insole of the present invention could be made with a hydrophilic agent on the upper surface which will be nearer to the foot of the wearer, while the lower surface which will be adjacent other parts of the footwear, could be made with a hydrophobic to keep the perspiration away from other parts of the footwear.

Office Partition and Office Component Fabrics

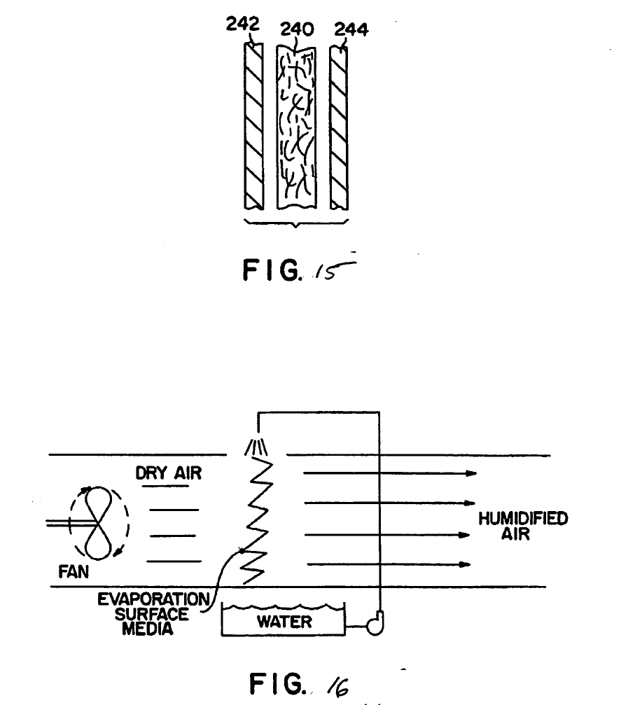

FIG. 15 illustrates a cross section of an office partition fabric having a filling layer 240, a fabric layer 242 on one side and a third layer 244 which may also be a fabric, or may be a solid material. Office type partitions walls can be portable or semi-portable divers of open area for personnel work stations and other assigned work and waiting areas for employees and clients. The fabric layers may be composed wholly of the hollow antimicrobial fibers, or may be blended with other synthetic or natural fibers to form a variety of fabrics and/or wall fillers. Partitions of this type are used in office factory, storage and customer service areas. They are provided with fabric surfaces (woven, knits, or non-woven) for aesthetic reasons, sound absorption and/or to cushion impacts. They may also be divided with internal fabric or loose fiber fills for cushioning, wall covering substrate support and sound and/or thermal insulation purposes. The antimicrobial agent is incorporated into the fibers in one or both of the outer layers 240 and 244. This can include fabrics for office, hospital, waiting area, classrooms, busses, cars, and the like and also curtains, upholstery, carpets and bedspreads. In addition to the antimicrobial agent, other materials can be added to the fibers such as pigments, fire retardants, color fixing agents, and UV resistant agents. Partitions are assembled, disassembled, moved and reassembled with some frequency. This and traffic around such partitions creates an environment for spread of airborne or contact transmitted disease, and partitions are frequently touched. This invention provides partition systems and other articles of the type described. An anti-static agent can be added to assist in dissipating static charges which create problems, for example, when computers are being used. The product remains intact when subjected to normal cleaning and can be assembled by being needle punched, resin bonded wet laid, thermo-bonded, and spun bond. In office environments there is the spillage of food and spills from office supply and janitorial materials and simple hand contact on wall surfaces. These and other environmental insults have the potential to leave residues that can be good substrates for the growth of bacteria, mold and other microbes. They can be in moist environments and the partitions are site for growth, and also from airborne microbes.

Car Wash Materials

Car wash materials, including shami type materials, in which the antimicrobial features last for the normal life of car wash cloths, for example, from 6 to 9 months. In car washes, many types of fabrics are used in the washing process. For instance, the automatic machines that wash cars use a variety of shaped fabrics to clean the car. In addition, cloths of various kinds are used in the waxing, dying, and finishing processes. Due to their continual contact with water, which itself is often recycled, these materials are often wet for long periods of time. This type of situation is very favorable to the growth of bacteria, fungi, and other microbes. As a result of the above, the use of antimicrobial fibers in the manufacture of materials used to clean cars in car washes is a desirable goal. These antimicrobial fiber-containing materials are useful in materials used by the automatic machinery and by individuals employed to clean the cars as well as in other ancillary materials. Specifically, the shaped fabrics used for automatically cleaning the car and the hand towels used to wax, dry, and otherwise finish the car are better products when these antimicrobial fibers are added to them. In manufacturing these materials, any of the embodiments described above could be used. Both the strength and resiliency of these materials is important given that they are used multiple times and are subject to being constantly in contact with water. Also, other modifications of the characteristics of these fibers and fabrics beyond that of adding antimicrobial agents, including the addition of agents to change the hydrophobicity, are useful in view of their constant contact with water. Thus, these antimicrobial materials that are manufactured to be used in car washes significantly reduce the growth of mold, mildew, and bacteria. By achieving this goal, odors associated with the long-term use of these materials is reduced. Also, the number of times they can be re-used before being discarded is increased, both because of the incorporation of antimicrobial fibers into these materials and the strengthening strategies indicated above. These characteristics also result in a significant costs savings in the operation of car washes. The hydrophilic and antimicrobial additives provide a hydrolysis-resistant surface that results in long-term protection against washings in boiling water and strong soaps, and also degreasers and chemical based cleaners. The antimicrobial synthetic fibers can further be blended with non-antimicrobial fibers such as cotton, wool, polyester, polypropylene, acrylic, nylon and the like, to provide antimicrobial finished fabrics that are able to withstand significant wear and washings and while maintaining their effectiveness.

Filters

Car wash water filters are more useful when the antimicrobial fibers are used in the making of such filters. Also batts and “brillo” type pads can be used which float, or are submerged in a recycled water storage tank, and the antimicrobial fibers included in them kill the microbes, which are in the tank. This is especially important in car washes, which recycle the wash water, which is the majority of car washes. In car washes, the water that is used to wash the cars and the associated materials for performing the washing and drying operations is often recycled water. However, there are several disadvantages to using recycled water. These include the dirt and odor-causing materials found in the water, including various bacteria, fungi, and other microbes. Because of the use of recycled water, very favorable conditions exist for the growth of bacteria, fungi, and other microbes. As a result of the above, the use of antimicrobial fibers in the manufacture of filter materials used to clean the recycled water before re-use in car washes is a desirable goal. These antimicrobial fiber-containing filters are useful in reducing the build-up of biological materials and films, both on the machinery employed to clean fabrics and other materials associated with the car wash process, due to the recycled water re-use. Specifically, the shaped fabrics used for automatically cleaning the car and the hand towels used to wax, dry, and otherwise finish the car are less prone to the development of bacterial and fungal films. They are also less likely to impart undesirable odors to the car itself. In addition, the recycled water itself would be less likely to impart any odors to the car. They assist in improving the air quality for customers as they drive through a car wash, and also for the employees. In manufacturing these materials, any of the embodiments described above could be used. Both the strength and resiliency of these materials is important given that they are used multiple times and are subject to the high pressures characteristic of filtering processes. Any number of filter shape designs could be used as appropriate to the step in the filtration that was being performed. In some instances, round filters would be appropriate whereas in other instances pleated or other shape filters would be appropriate, all depending on the pressure and volume characteristics of the recycled water flow. Also, the batts mentioned above can be used in the recycled water storage tanks or sumps to assist in cleaning the water by killing microbes and fungi. Anti-odor additives may be particularly useful in this application given the use of recycled water. Thus, these antimicrobial car wash filters and batts significantly reduce the growth of mold, mildew, and bacteria in the recycled water and on car wash materials. By achieving this goal, odors associated with the long-term use of recycled water and these materials would be reduced. Also, the number of times the recycled water and the car wash materials could be re-used before being discarded could be increased. The ability to re-use recycled water several additional times because these types of filters and/or batts are employed in the recycle process would results in a significant costs savings in the operation of car washes.

Institutional Products and Home Furnishings

Institutional products and home furnishings, such as bed sheets, pillow cases, mattress pads, blankets, towels, drapes, bedspreads, pillow shams, carpets, walk-off mats, napkins, linens, wall coverings, upholstered furniture, liners, mattress ticking, mattress filling, pillow filling, carpet pads, upholstery fabric and the like, are significantly improved when made using, at least in part, the antimicrobial fibers described above. Further details of these institutional products and home furnishings are provided below; Mattress pads ½″ to 1″ in thickness may be made, for example, as set forth in Example 1 above. The web can be air laid and the binder fiber melts in an oven.

Bed sheets and pillowcases can be made of antimicrobial fiber. They can be constructed using low melt binder fiber blended in at levels of 1 to 20%. The binder fiber can be blended with other fibers such as cotton, wool, polyamides, viscose, flax, acrylic, or polyester. The low melt binder fiber contains levels of the active antimicrobial ingredient ranging from 0.25% to 5%. Fiber properties are from 0.7 denier through 25 denier with cut lengths ranging from 1 mm to 180 mm.

The antimicrobial fibers are used to spin yarn in cotton counts ranging from 4's to 80's. Sheets and pillowcases may be woven or knitted. Yarns used to weave the bed sheets/pillowcases, containing the antimicrobial treated fibers, may be used only in the warp direction, or the filling direction, or may be used in both.

Some sheets and pillowcases have been made using 1-15% antimicrobial fiber in the fabric, which are 1.5-3.5 denier, 1½″ staple length and in which 15% of the filling yarn is antimicrobial. For example, they can have 15% antimicrobial fiber, 35% cotton and 50% untreated polyester.

PETG is blended with the cotton, and is heated, it does not ball up but wicks along the other fibers. The cross section becomes thinner as the PETG flows. For loose knit fabrics 15-20% antimicrobial fiber is useful to kill the microbes, whereas for flat woven fabric there can be 10% or less antimicrobial fiber to kill microbes.

The same fabric can be used in bed sheets and for medical scrubs. Woven fabric is desized to remove starch from the warp yarns. High loft batting may be used to stuff the mattress pad. In one example, the fiber may be made with all PET in 6½ oz per square yard, 6 denier blended with 6 denier regular while.

The antimicrobial fibers are used for the top and bottom layers of the pads which are sealed or connected to each other along their perimeters. This can be by sewing with thread or in some other suitable manner. The center is filled with a batting material which includes 15% antimicrobial fiber produced as described below. The top and bottom layers are woven fabric which is made from yarn which contains 15% antimicrobial fiber produced as described below.

It has been found that when these fabrics are dyed, the dyeing process can have the effect of blocking the antimicrobial action. However, in accordance with the present invention this problem is resolved by using hot water soaks or washes which rejuvenates the fiber's antimicrobial agents.

Antimicrobial fibers can be used to make materials for a variety of applications in which it is necessary or desirable to reduce bacterial and fungal growth and their resultant odor. Specifically, in institutional environments, these materials can be used in support substrates for furnishings. In these situations, these support materials are subject to a variety of environmental insults that can cause the growth of bacteria, fungi, and other microbes. These include the spillage of food and its seepage inside furnishings and spills from janitorial materials. These and other environmental insults have the potential to leave residues that can be good substrates for the growth of bacteria, mold, and other microbes. Therefore, unsanitary conditions can occur along with the associated bad odor, both of which can contribute to patient sickness and allergy, a deterioration of patient morale, and sick building syndrome, in general.

As a result of the above, the use of antimicrobial fibers in the manufacture of support substrates for institutional furnishings is a desirable goal. These antimicrobial fiber-containing support substrates are useful in reducing the build-up of biological materials and films, thus reducing associated patient discomfort and environmental contamination. Specifically, the antimicrobial-fiber containing support substrates could be coated with polyvinyl chloride (PVC) or laminated to woven or knit fabrics in the construction of institutional furnishings.

In manufacturing the furnishing type materials, both the strength and resiliency of these materials is important given that they must stand up to a variety of environmental insults, frequent moves, and varying storage conditions. They must also be strong enough to act as supporting members of the furnishings themselves.

Color pigments may be added to these antimicrobial fibers in order to provide the desired coloration for finished fabrics and materials. Similarly to the above antimicrobials, these pigment materials can be added such that the pigments are encapsulated in the polymers that are used to make these fabrics. By using this method of coloring the fibers, materials and fabrics made from these colored fibers are color-fast and do not leach out their color during washing, thus significantly reducing fading during wear and washing. In addition, since the need for conventional dyeing techniques can be reduced or eliminated, the disposal of environmentally damaging dye materials is avoided. This, in and of itself, can reduce the costs of manufacturing finished colored fabrics due to the elimination of the manufacturing infrastructure and associated personnel needed to process residual dye effluents.

In a similar fashion to antimicrobial agents and color pigments, a variety of other additives that are used for various purposes can be combined with the polymers during or after fiber formation and extrusion. For example, additives that protect against damage from UV light may be added to the fiber polymer or coated onto it so that the fabrics and materials formed are resistant to the fading of colors and UV damage generally. Both flame-resistant and -retardant agents can also be added to the fibers of this invention in a manner similar to that described for UV protecting agents. In this way, the fabrics and materials formed can be made resistant to fire. Anti-stain agents can also be added to the fibers or resultant fabrics in the above manner.

In addition, the fibers can be made either hydrophilic or hydrophobic as desired by mixing other agents into the fiber polymers or applying them to the fiber surface. By modifying the wetability characteristics of the fibers, they can be made more useful for various applications. For example, hydrophilic fibers are effective in applications in which one wants the antimicrobial fabric or material to more easily absorb water, such as when the fabric is designed to absorb solutions containing bacteria and fungi and other microbes. Alternatively, hydrophobic fibers are effective in applications in which one wants to avoid the absorption of such solutions, such as in the manufacture of clothing, in general, and in work clothes, in particular.

The antimicrobial agents can also be added to low-melt polymer fibers that can be activated and melted during fabric production by raising the temperature, thus spreading the antimicrobial agents throughout the fabric when the low-melt fibers melt and coat the interstitial intersections of the other fibers. By varying the amount of antimicrobial-containing low-melt fiber regionally and/or by varying the amount of antimicrobial agent in these low-melt fibers, a fabric or material can be produced that has a purposely designed regional variation in antimicrobial effectiveness throughout.

Specifically, the latter situation can be achieved by using an amorphous binding fiber such as PETG, which can be blended into yarns and with other fibers to form fabrics and materials. After heat activation, the PETG fibers melt, wetting the surface of the surrounding fibers and settling at the junctions of other heat-stable fibers. In this way, solidified drops of PETG form at these junctions and bind the fibers together while spreading the antimicrobial agent throughout the fiber. Because of the excellent wetting characteristics of PETG, the antimicrobial agent can be uniformly distributed throughout the fabric. These methods of activating PETG fibers may also be used to additionally distribute pigments and the other additives described above throughout the finished fabrics and materials.

The binder fiber carrier containing polymers and antimicrobial additives can be blended with non antimicrobial fibers such as cotton, wool, polyester, acrylic, nylon, PTT, 3GT, rayon, modified rayon, and acetate to form antimicrobial finished fabrics. Thus, an antimicrobial finished fabric is produced that is able to withstand significant wear and washings and maintain its effectiveness.

A typical example of this embodiment is a fiber using PETG polymer with a silver zeolite additive to blend with cotton at concentrations up to 10 percent by weight to produce a bed sheet. The binder fiber is activated in the drying cycle of the final bleaching operation or other heat operation. The PETG then melts and wets the surface of the cotton fibers to carry the antimicrobial property to the entire sheet with an added benefit of increasing strength and reducing pilling.

Athletic Wear

Athletic wear clothing and liners, including athletic wear liners can be made from the hollow antimicrobial fibers wholly or partly, and as binder fibers in staple and/or filaments, optionally with antimicrobial properties. Athletic wear is subject to the accumulation of bacteria, fungi, and associated odors that can proliferate in the presence of sweat and other bodily secretions that result from strenuous exercise in this type of clothing. This type of product may be made using antimicrobial fibers, and which for some applications are provided with a layer which touches the skin and wicks away the sweat to make a more comfortable garment (or liner) and this type of article benefits from the use of antimicrobial fibers in at least one layer. They can include T-shirts, crotch liners, bicycle pants and shirts, sweat suits, athletic supporters, stretch pants, long underwear, and athletic socks. Because this type of clothing is constantly and intermittently being soaked with sweat and brought into contact with dirt and associated materials, they are subject to bacterial and fungal growth as well as to the development of associated odors. By manufacturing this clothing with lining materials made, at least partially, of the antimicrobial fibers of this invention, growth of microbes could be reduced. In addition, the exacerbation of microbial growth and resultant odor production upon storage of this type of clothing in bags over time could be reduced. These antimicrobial fiber-containing clothing is useful in reducing the growth of bacteria, fungi, and other microbes once soaked with sweat, thus reducing associated odors and the discomfort of the individual. Specifically, the antimicrobial-fiber containing fabrics may be used in the interior linings of shirts and pants or shorts, such as those used in running and bicycling. These antimicrobial fibers may also be used in the manufacture of athletic clothing that does not have linings. This type of athletic clothing is then able to be used for long periods of time while maintaining its antimicrobial and anti-odor properties because of its resistance to multiple washings. In addition, the methods described above could also be used to produce clothing dyed in a variety of colors that would possesses the characteristics of inhibiting microbial growth and its associated odors, thus increasing its versatility.

Mop Head Fabrics

Mop head fabrics incorporating hollow antimicrobial fibers can comprise fibers in yarns, knitted fabrics, woven fabrics or non-woven fabrics. Mop head fabrics are subject to bacterial and fungal growth due to their constantly being wetted upon use, and are left wet in storage and allowed to air-dry. This constant wetting also causes the development of odors and the eventual deterioration of the integrity of the mop head materials themselves. Mop heads can transfer bacteria and fungi from one area to another and thus can be the cause of significant collections of microbes and fungi. Thus, these mop head fabrics made from antimicrobial materials significantly reduce the growth of mold, mildew, and bacteria. By achieving this goal, odors associated with the long-term use of these materials are reduced. Also, the number of times they may be re-used before being discarded is increased, both because of the incorporation of antimicrobial fibers into these materials and the strengthening strategies indicated above. These characteristics also result in a significant costs savings in the use of mop heads in industrial settings.

Medical Wipes

The antimicrobial hollow fibers may also be used in medical wipes. Specifically, the antimicrobial-fiber containing fabrics may be used in both the covering fabric and the water absorbent interior material. In this way, both surface and interior protection can be achieved. In addition, these materials could also be manufactured as reusable wipes because the antimicrobial effect of the fibers of this invention are resistant to multiple washings. Thus, a significant cost savings could be realized in the purchasing of supplies in a variety of institutional settings, including hospitals and nursing homes.

The finished product may be constructed of nonwoven, knit, woven or other process. It may also be treated or pre-moistened with a topical treatment such as a soap solution or other additive. The finished product can be produced from any combination of natural or synthetic fiber in addition to the antimicrobial fibers. The wipe cloth may be unitary or combined or laminated to some other fabric.

In manufacturing these materials, any of the embodiments described above or below can be used. Both the strength and resiliency of these materials is important given that they must withstand the cleaning of multiple surfaces.

In one multi-layer embodiment, there is a skin contacting layer which contains the antimicrobial fibers, an absorbent layer adjacent to the first layer and which contains a cleaning solution, a non-permeable layer adjacent the absorbent layer to prevent the user being contacted with the solution or by any of the products from a wound, and a tab attached to the non-permeable layer as a handle for the user.

Dust Masks

Dust masks are vulnerable to the capture and seeding of bacteria and fungi. They can provide hospitable sites for the protected growth and the inhalation/exhalation of microbes. These products benefit from having anti-bacterial and anti-fungal agents incorporated into them. Dust masks may be of a nonwoven construction of antimicrobial fibers (at least in part) and may be covered on one or both sides with a fabric layer. Such masks which can have or provided antimicrobial containing filters are useful in reducing the build-up of biological materials on the dust mask which could be inhaled by the user.

Fibrous Media

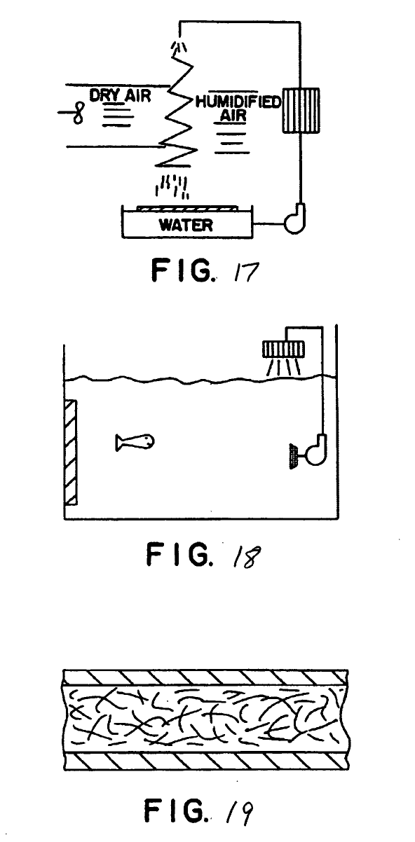

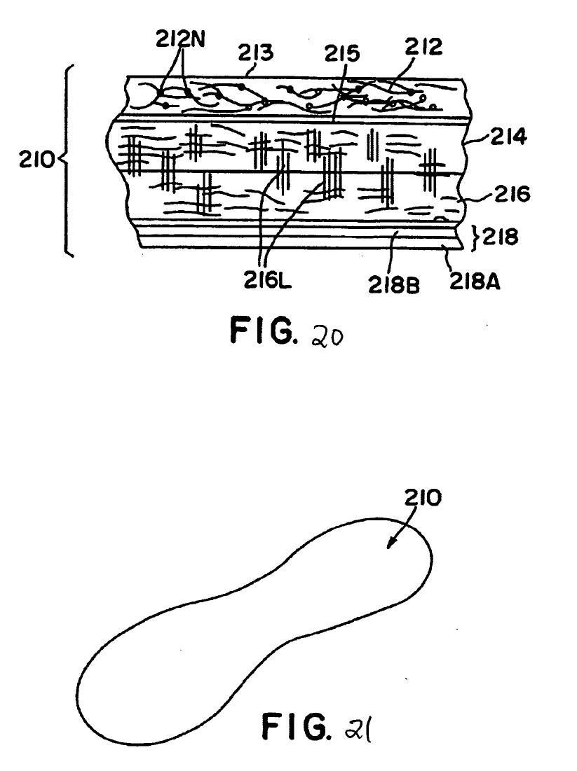

Humidifier evaporation surface media introduces an antimicrobial fiber into the evaporation surface media for humidifiers. Such a media prevents the growth of mold, mildew, bacteria, and fungi on the media. Preventing such growth reduces or eliminates the “musty smell” currently experienced when such devices are started up to humidify home or office environments. It reduces or prevents the growth of organisms in humidifier systems to prevent odor and bacterial growth. The media may be made of a nonwoven fibrous material made at least in part of the antimicrobial fibers disclosed herein. FIG. 16 presents a schematic view of a humidifier evaporation surface media, which is made at least in part of antimicrobial fibers, used to humidify air. FIG. 17 shows a humidifier pad which could float on the surface of a tank, be attached to the bottom or sides of the tank, or in the suction or discharge sides of the circulation pump, and it is made at least in part of the antimicrobial fiber disclosed herein. FIG. 18 shows a “fish tank” circulation/aeration system. An antimicrobial pad or filter is on the suction or discharge side of the pump or attached to the bottom on the sides of the tank. This helps prevent the growth of microbes in recirculation systems and tanks which can not use chemicals or in which it is desired not to use chemicals. This and other uses for antimicrobial fibers in different environments show that a person working, for example, in a moldy or dirty environment would want as much assistance as possible in a respirator or filter or mask. Also, one wants the antimicrobial agent to remain in the fiber and not be inhaled by the user.

Boat Bilge Pads

Boat bilge antimicrobial pads can be made at least in part with antimicrobial fibers can be used in a filter in the system or can be used in a manner similar to that of the car wash filter in pads which are placed into the water storage tank to kill bacteria in the water.

Laundry Bags

Laundry bags can be made at least in part of antimicrobial fibers as described herein to reduce odors and to kill bacteria which may be present in the bags.

Apparel

Apparel can be made using antimicrobial fiber as described elsewhere herein.

Insoles

A further embodiment of practice of the invention is shown in FIGS. 20 and 21 wherein an insertable innersole 210 for shoes and boots is made up of multi-layers indicated in FIG. 20. The layering is indicated before heating and pressing this laminate to form a bonded construction. The innersole has antimicrobial that are available in the as fully manufactured product and, as in other embodiments of the invention described above, are provided in a cost efficient way.

A top layer 212 of the laminate is made of a non-woven or woven array of fibers, preferably of polyester, has an overall weight of 2.5 to 6.0 oz. per square yard and includes some 5-25% of its weight as antimicrobial hollow fibers incorporating zeolites of silver or other antimicrobial dispersed substantially uniformly in the layer. In eventual processing the surface 213 gets treated by embossing, ultrasonic bonding and/or other modification and the layer as a whole is heated (along with heating and pressing the laminate as a whole) to effect, among other things, bonding of fibers at many cross over points (nodes) 212N in a manner well known in the art to effect densification and strength while retaining substantial porosity and moisture vapor permeability through the layer.

The next major layer 214 is made of thermo-formable polymer blends of hollow antimicrobial fibers and non-hollow or hollow non-antimicrobials fibers, preferably polyesters and/or co-polyesters, the former incorporating antimicrobial agents, to form a layer of weight 2.5-9.0 oz. per square yard. The layer is non-woven needle-punched fabric with some distinct fiber orientation in the lateral direction within layer 214 itself and with punched through fibers from the next lower layer as described below. This layer 214 is bonded to layer 212 by a an adhesive web of scrim or mesh form of 15-30 gm per sq. meter weight (very diaphanous) and made of polyester, polyolefins (polethylene, polypropylene, etc.), polyamide or other fiber materials and in the course of laminate heating and pressing becomes an effective bonding agent to bond layers 212, 214 securely to prevent delamination in service use.

The next major layer 216 is designed as a moisture storage (and eventual off-gassing) layer with high surface area fibers, including 20-50 weight percent of 4 DG lobed or grooved fibers of polyester or other fiber material of a type well known per se, 50-60 weight percent of normally surfaced polyester fibers and 5 to 25 weight percent of fibers containing antimicrobial agents. The fibers are preferably normally surfaced but could also be made of grooved form, consistent with the missions of antimicrobial agent carriage and access. The layer as a whole weighs 4-12 oz. per sq. yard and is bonded to layer 214 by deep needle-punching fibers of layer 216 into layer 214 using barbed felting needles to establish lateral wicking paths as indicated, e.g., at 216L.

The final layer 218 is a co-extruded two part plastic film with a barrier sub-layer portion 218A and a bonding sub-layer portion 218B, each such portion being 25-100 microns thick and made of A/B combinations of, e.g., polypropylene/polyethylene, polypropylene/polyester, polyropylene/polyamide, etc.

When the laminate is heated and pressed under state of the art conditions for molding such materials the layer 214 becomes highly densified and entraps the lateral fibers 216L to secure layers 214, 216 together while bonding layers 215 and 218B secure the outermost layers to the laminate.

The tough upper layer 212 resists cracking and shedding under the impact of direct user contact and flexing in use or when removed from a shoe but allows free flow of moisture vapor which is wicked through layer 214 to moisture storage layer 216 in an efficient way and retained there because of the bonded on moisture barrier 218A so that odor doesn't go beyond the innersole to any substantial degree. The overall result is an odor absorbing innersole of fibrous material that provides necessary cushioning in a slim profile that can fit comfortably in an athletic or dress shoe or boot or moccasin/loafer. No foam materials or charcoal adsorbents or the like need be used. Moisture can be absorbed in the present product and retained with high destruction of odor causing microbes and the moisture can desorb gradually with lowered concentrations of odor causing microbes with two to three order of magnitude reduction.

Nautical Fabrics

Nautical fabrics can be made at least in part using the antimicrobial fibers of the present invention and are particularly useful for this type of application in which the fabrics are constantly wet and subject to mildew.

Moldable Laminates

Moldable laminates for footwear are described in more detail below. The present invention provides a binding agent in a nonwoven product in which the binding agent is a thermoplastic hollow binder fiber. The binder fiber is thermally activated in order to bind (stiffen) the nonwoven portion of the product. Since this is produced with 100% thermoplastic components allows for easy recycling. The product is a thermal moldable impact resistant stiffener for footwear applications such a counter or box toe.

A 100% thermoplastic, stiff reinforcing multiple laminate structure which can be moldable into complex, compound shapes and bondable via a thermoplastic hot melt adhesive to a carrier surface to be reinforced to provide a tough, water resistant reinforcement, usable for instance in stiffening applications as a footwear counter or box toe reinforcement element that is recyclable into itself. The fabric layer is in part geometrically locked into the tough thermoplastic resin layer.

The needle punched nonwoven is manufactured from hollow staple fibers or blends. The nonwoven utilizes a combination of PET fibers and PETG or other copolymer or homopolymer fibers that act as a binding agent for PET. The staple fiber is 4-15 denier and 38 to 76 mm in length.

The thermoplastic components of the product are either miscible or mechanically compatible so as to allow for homogenization and complete recyclability of scrap material. The binder fibers have a low melting temperature, and the fiber portion of the product is prepared as disclosed elsewhere herein.

It will now be apparent to those skilled in the art that other embodiments, improvements, details, and uses can be made consistent with the letter and spirit of the foregoing disclosure and within the scope of this patent, which is limited only by the following claims, construed in accordance with the patent law, including the doctrine of equivalents.

1. An antimicrobial fiber, comprising:
a fiber having a thermoplastic, polymeric wall defining one or more axially extending voids within the fiber; and
antimicrobial particles interspersed within the wall.

2. The fiber of claim 1, wherein the antimicrobial particles comprise organic or inorganic particles.

3. The fiber of claim 1, wherein the wall has a thickness in the range of about 1.73 to 2.25 times the nominal size of the antimicrobial particles.

4. The fiber of claim 1, wherein the antimicrobial particles comprise one or more inorganic additive selected from the group consisting of copper, silver, tin, and zinc.

5. The fiber of claim 4, wherein the one or more inorganic additives are incorporated in a carrier selected from the group consisting of zirconium phosphate, zeolites, and dissolvable glass.

6. The fiber of claim 1, wherein the antimicrobial particles comprise zeolites of silver particles.

7. The fiber of claim 1, wherein the antimicrobial particles comprise triclosan particles.

8. The fiber of claim 1, wherein the fiber wall is composed of a polymer selected from the group consisting of polyester, nylon (polyamid), rayon, lyocell, polypropylene, polyethylene, aramid, acrylic, PCT, PETG, Co-PET, PTT, 3GT, and polyamide 6 or 6,6.

9. The fiber of claim 1, further comprising an additive interspersed in the wall selected from the group of materials consisting of pigments, anti-odor compounds, fire-retardant materials, hydrophilic materials, hydrophobic materials, UV additives, and/or anti-stain materials.

10. The fiber of claim 1, wherein the one or more axially extending voids extend the entire length of the fiber.

11. The fiber of claim 1, wherein the fiber is continuous filament.

12. The fiber of claim 1, wherein the wall includes an axially extending discontinuity along the length of the fiber.
